SEQUENCE LISTING (6 sequences)

SEQ ID NO: 1 - protein, 333 aa, Mus musculus
MKLGRAALALLLLAPCVVRAVEPISLSLALAGVLTTYISYPRLYCLFAECCGQMRSLSREALQKDLDNKLFGQHLAKKVILNAVSGFLSNPKPKKPLTLSLHGWTGTGKNFASKIIAENIYEGGLNSDYVHLFVATLHFPHASNITQYKDQLQMWIRGNVSACARSIFIFDEMDKMHAGLIDAIKPFLDYYDVVDEVSYQKAIFIFLSNAGAERITDVALDFWKSGKQREEIKLRDMEPALAVSVFNNKNSGFWHSSLIDRNLIDYFVPFLPLEYKHLKMCIRVEMQSRGYEVDEDIISKVAEEMTFFPKEEKVFSDKGCKTVFTKLDYYLDD

SEQ ID NO: 2 - DNA, 1452 bp, Mus musculus
  source 1..1452: mol_type = other DNA
gcggtgcgcggggtacggtgggcgctgggcgggcgctgggcgcgcaaggtgcgcgggtccggttatgaagcttggccgggccgctctggccctgctgctgctggcgccgtgcgtggttcgtgcggtggagcccatcagcctgagtctggccctggccggcgtactcaccacctatatctcctaccctcgtctctactgcctcttcgccgagtgctgcggccagatgcggagcctcagccgggaggcgctgcagaaagatctggataacaagctctttggacagcatcttgcaaaaaaagtcatcctaaacgccgtgtctggtttcctaagcaacccgaagcccaagaagccccttaccctctctctgcacgggtggacgggcaccggcaaaaacttcgccagcaagatcatcgcggagaatatttacgagggcggactgaacagtgactatgtacacctgtttgtggccacgctacacttcccccacgcctctaacatcacacagtataaggaccagttacagatgtggatcagaggcaacgtgagcgcctgtgctcgctccatcttcatctttgatgagatggacaagatgcatgccggcctcatcgacgccatcaagcctttcctagactattacgatgtggtagatgaggtctcctatcagaaagccatcttcatcttcctcagcaatgcaggggcagagaggatcacagacgtggctctggatttctggaaaagtgggaagcagagggaagaaatcaagctcagagacatggagcccgccctggccgtgtcggtcttcaataacaagaacagtggcttctggcacagcagcctcattgaccggaacctcatagattattttgtccccttcctgcccctggagtacaagcacctgaaaatgtgcatcagagtggagatgcagtcccgaggctatgaagtagatgaggacatcatcagcaaggtagcggaagagatgacgttcttccccaaggaggagaaggtcttctctgacaagggctgcaagactgtgttcaccaagctggactactacctggatgactgaggccggctgccagggctgtgtcagtccgctcctccccagaagaggcttggacacttcctgctgaggttagcgggagaatcctgtttctgctcctcagagcctgcaggtgaccgcgctgtagggaccacagatgcaacatggacctgaagctcctggagcctccagagatgaaagcagctcccagcattctggacttgtgatttttttaaaattatgttttaatttcaggtgtttctgttttgaggcagaacaagtaagttttactgcaaatgtgataacgttttatttaaaatggtttttaatactataagaagctttcagcggtggctttgtgtgtcttttgaaactgttggatgacattaccagtatttctacttcagaaaccc

SEQ ID NO: 3 - DNA, 1002 bp, Mus musculus
  source 1..1002: mol_type = other DNA
atgaagcttggccgggccgctctggccctgctgctgctggcgccgtgcgtggttcgtgcggtggagcccatcagcctgagtctggccctggccggcgtactcaccacctatatctcctaccctcgtctctactgcctcttcgccgagtgctgcggccagatgcggagcctcagccgggaggcgctgcagaaagatctggataacaagctctttggacagcatcttgcaaaaaaagtcatcctaaacgccgtgtctggtttcctaagcaacccgaagcccaagaagccccttaccctctctctgcacgggtggacgggcaccggcaaaaacttcgccagcaagatcatcgcggagaatatttacgagggcggactgaacagtgactatgtacacctgtttgtggccacgctacacttcccccacgcctctaacatcacacagtataaggaccagttacagatgtggatcagaggcaacgtgagcgcctgtgctcgctccatcttcatctttgatgagatggacaagatgcatgccggcctcatcgacgccatcaagcctttcctagactattacgatgtggtagatgaggtctcctatcagaaagccatcttcatcttcctcagcaatgcaggggcagagaggatcacagacgtggctctggatttctggaaaagtgggaagcagagggaagaaatcaagctcagagacatggagcccgccctggccgtgtcggtcttcaataacaagaacagtggcttctggcacagcagcctcattgaccggaacctcatagattattttgtccccttcctgcccctggagtacaagcacctgaaaatgtgcatcagagtggagatgcagtcccgaggctatgaagtagatgaggacatcatcagcaaggtagcggaagagatgacgttcttccccaaggaggagaaggtcttctctgacaagggctgcaagactgtgttcaccaagctggactactacctggatgactga

SEQ ID NO: 4 - protein, 332 aa, Homo sapiens
MKLGRAVLGLLLLAPSVVQAVEPISLGLALAGVLTGYIYPRLYCLFAECCGQKRSLSREALQKDLDDNLFGQHLAKKIILNAVFGFINNPKPKKPLTLSLHGWTGTGKNFVSKIIAENIYEGGLNSDYVHLFVATLHFPHASNITLYKDQLQLWIRGNVSACARSIFIFDEMDKMHAGLIDAIKPFLDYYDLVDGVSYQKAMFIFLSNAGAERITDVALDFWRSGKQREDIKLKDIEHALSVSVFNNKNSGFWHSSLIDRNLIDYFVPFLPLEYKHLKMCIRVEMQSRGYEIDEDIVSRVAEEMTFFPKEERVFSDKGCKTVFTKLDYYYDD

SEQ ID NO: 5 - DNA, 2080 bp, Homo sapiens
  source 1..2080: mol_type = other DNA
gcaccggttcgcggtcggcgcgagaacaagcagggtggcgcgggtccgggcatgaagctgggccgggccgtgctgggcctgctgctgctggcgccgtccgtggtgcaggcggtggagcccatcagcctgggactggccctggccggcgtcctcaccggctacatctacccgcgtctctactgcctcttcgccgagtgctgcgggcagaagcggagccttagccgggaggcactgcagaaggatctggacgacaacctctttggacagcatcttgcaaagaaaatcatcttaaatgccgtgtttggtttcataaacaacccaaagcccaagaaacctctcacgctctccctgcacgggtggacaggcaccggcaaaaatttcgtcagcaagatcatcgcagagaatatttacgagggtggtctgaacagtgactatgtccacctgtttgtggccacattgcactttccacatgcttcaaacatcaccttgtacaaggatcagttacagttgtggattcgaggcaacgtgagtgcctgtgcgaggtccatcttcatatttgatgaaatggataagatgcatgcaggcctcatagatgccatcaagcctttcctcgactattatgacctggtggatggggtctcctaccagaaagccatgttcatatttctcagcaatgctggagcagaaaggatcacagatgtggctttggatttctggaggagtggaaagcagagggaagacatcaagctcaaagacattgaacacgcgttgtctgtgtcggttttcaataacaagaacagtggcttctggcacagcagcttaattgaccggaacctcattgattattttgttcccttcctccccctggaatacaaacacctaaaaatgtgtatccgagtggaaatgcagtcccgaggctatgaaattgatgaagacattgtaagcagagtggctgaggagatgacatttttccccaaagaggagagagttttctcagataaaggctgcaaaacggtgttcaccaagttagattattactacgatgattgacagtcatgattggcagccggagtcactgcctggagttggaaaagaaacaacactcagtccttccacacttccacccccagctcctttccctggaagaggaatccagtgaatgttcctgtttgatgtgacaggaattctccctggcattgtttccaccccctggtgcctgcaggccacccagggaccacgggcgaggacgtgaagcctcccgaacacgcacagaaggaaggagccagctcccagcccactcatcgcagggctcatgattttttacaaattatgttttaattccaagtgtttctgtttcaaggaaggatgaataagttttattgaaaatgtggtaactttatttaaaatgatttttaacattatgagagactgctcagattctaagttgttggccttgtgtgtgtgtttttttttaagttctcatcattattacatagactgtgatgtatctttactggaaatgagcccaagcacacatgcatggcatttgttccacaggagggcatccctggggatgtggctggagcatgagccagctctgtcccaggatggtcccagcggatgctgccaggggcagtgaagtgtttaggtgaaggacaagtaggtaagaggacgccttcaggcaccacagataagcctgaaacagcctctccaagggttttcaccttagcaacaatgggagctgtgggagtgattttggccacactgtcaacatttgttagaaccagtcttttgaaagaaaagtatttccaacttgtcacttgccagtcactccgttttgcaaaaggtggcccttcactgtccattccaaatagcccacacgtgctctctgctggattctaaattatgtgaattttgccatattaaatcttcctcatttatactattatttgttacgttcaatcagaatccccgaaacctcctataaagcttagctgccccttctgaggatgctgagaacggtgtctttctttataaatgcaaatggctaccgttttacaataaaattttgcatgtgcca

SEQ ID NO: 6 - DNA, 999 bp, Homo sapiens
  source 1..999: mol_type = other DNA
atgaagctgggccgggccgtgctgggcctgctgctgctggcgccgtccgtggtgcaggcggtggagcccatcagcctgggactggccctggccggcgtcctcaccggctacatctacccgcgtctctactgcctcttcgccgagtgctgcgggcagaagcggagccttagccgggaggcactgcagaaggatctggacgacaacctctttggacagcatcttgcaaagaaaatcatcttaaatgccgtgtttggtttcataaacaacccaaagcccaagaaacctctcacgctctccctgcacgggtggacaggcaccggcaaaaatttcgtcagcaagatcatcgcagagaatatttacgagggtggtctgaacagtgactatgtccacctgtttgtggccacattgcactttccacatgcttcaaacatcaccttgtacaaggatcagttacagttgtggattcgaggcaacgtgagtgcctgtgcgaggtccatcttcatatttgatgaaatggataagatgcatgcaggcctcatagatgccatcaagcctttcctcgactattatgacctggtggatggggtctcctaccagaaagccatgttcatatttctcagcaatgctggagcagaaaggatcacagatgtggctttggatttctggaggagtggaaagcagagggaagacatcaagctcaaagacattgaacacgcgttgtctgtgtcggttttcaataacaagaacagtggcttctggcacagcagcttaattgaccggaacctcattgattattttgttcccttcctccccctggaatacaaacacctaaaaatgtgtatccgagtggaaatgcagtcccgaggctatgaaattgatgaagacattgtaagcagagtggctgaggagatgacatttttccccaaagaggagagagttttctcagataaaggctgcaaaacggtgttcaccaagttagattattactacgatgattga

(19) United States
(12) Patent Application Publication
(10) Pub. No.: US 2026/0157352 A1
(43) Pub. Date: Jun. 11, 2026

(54) DYSTONIA MOUSE MODELS WITH OVERT DYSTONIA

(71) Applicant: University of Florida Research Foundation, Incorporated, Gainesville, FL (US)

(72) Inventors: Yuqing LI, Gainesville, FL (US); David E. VAILLANCOURT, Gainesville, FL (US); Hong XING, Gainesville, FL (US); Pallavi GIRDHAR, Gainesville, FL (US)

(21) Appl. No.: 19/312,538

(22) Filed: Aug. 28, 2025

Related U.S. Application Data
(60) Provisional application No. 63/688,331, filed on Aug. 29, 2024.

(51) Int. Cl. A01K 67/0278; C12N 15/85

(52) U.S. Cl. CPC A01K 67/0278; C12N 15/8509; A01K 2217/07; A01K 2217/15; A01K 2227/105; A01K 2267/035; C12N 2015/8536

(57) ABSTRACT

Provided herein are transgenic mouse models of DYT1 dystonia with partially conditional knock-in of the DYT1 dystonia trinucleotide deletion (ΔGAG) in exon 5 of the mouse Tor1a gene (also known as Dyt1). Also provided are methods of generating these mice and methods of their use for preclinical screening of potential dystonia therapeutics.

### CROSS-REFERENCE TO RELATED APPLICATIONS

This application claims the benefit of U.S. Provisional Application No. 63/688,331, filed Aug. 29, 2024, which is herein incorporated by reference in its entirety for all purposes.

### GOVERNMENT INTERESTS

This invention was made with government support under Grant No. R01 NS075012 and Grant No. R01 NS129873, awarded by the National Institutes of Health; and by Grant No. W81XWH-21-1-0198, awarded by the Defense Health Agency. The government has certain rights in the invention.

### REFERENCE TO A SEQUENCE LISTING SUBMITTED AS AN XML FILE

The Sequence Listing written in file 634647SEQLIST.xml is 12,288 bytes, was created on Aug. 20, 2025, and is hereby incorporated by reference.

### BACKGROUND

DYT1 dystonia is a neurological movement disorder that results in abnormal postures, uncontrollable repetitive movements, and muscle contractions. DYT1 dystonia is an early-onset inherited dystonia caused by a trinucleotide deletion (ΔGAG) in the DYT1 gene (also known as TOR1A) that encodes the protein torsin-1A. DYT1 dystonia symptoms usually start in childhood or early adolescence (before the age of 26), usually around age 12. Currently, there is no cure for dystonia. The only available treatment options alleviate symptoms, and their effectiveness is variable from patient to patient. Oral medications for dystonia include anticholinergic agents (e.g., trihexyphenidyl), GABAergic agents (e.g., benzodiazepine), and dopaminergic agents (e.g., levodopa). Other treatments include regular botulinum toxin injections, which relax and reduce the contraction of muscles and prevent acetylcholine release. Some patients benefit from surgical intervention through deep brain stimulation, usually targeting the globus pallidus internus (GPi). However, a need remains for more effective or even curative treatments for DYT1 dystonia.

In this regard, preclinical animal models that accurately reproduce the disease phenotype of DYT1 dystonia are essential for the development of new therapeutic agents. Although several models have been developed to date, the vast majority of these models fail to display an overt dystonia phenotype consistent with human disease. Those animal models that do exhibit over dystonia, unfortunately, also display additional phenotypes that are inconsistent with human disease (e.g., weight loss, seizures, neurodegeneration, etc.) and would compromise any interpretation of results from preclinical testing, eliminating them as viable model systems for use in preclinical development of therapeutic agents to treat DYT1 dystonia. Thus, a need exists for better models of DYT1 dystonia that can reproduce the overt dystonia observed in human disease, without the extraneous phenotypes present in currently available models.

### SUMMARY

Provided are genetically modified mice whose genomes comprise a genetically modified Tor1a locus. The genetically modified Tor1a locus comprises: (i) a Tor1a allele comprising a ΔGAG deletion associated with DYT1 dystonia in exon 5; and (ii) an inducible Tor1a allele comprising (a) loxP sites flanking a wild type exon 5 of Tor1a and (b) a downstream copy of exon 5 comprising the ΔGAG deletion associated with DYT1 dystonia.

Also provided are progeny mice obtained by breeding a mouse comprising the genetically modified Tor1a locus with a transgenic mouse whose genome comprises a Cre recombinase. In the cells of the progeny mouse that express the Cre recombinase, the wild type copy of exon 5 of Tor1a in the inducible allele is deleted and the downstream copy of exon 5 comprising the ΔGAG deletion is expressed.

In some such mice, the Cre recombinase is expressed under the control of a tissue-specific promoter. Optionally, the tissue-specific promoter is Emx1. In some such progeny mice, the mouse exhibits overt dystonia. In some such progeny mice, the mouse does not exhibit impaired growth, reduced weight, seizures, neurodegeneration, or reduced lifespan. Optionally, the tissue-specific promoter is Rgs9. In some such progeny mice, the mouse exhibits overt dystonia. In some such progeny mice, the mouse does not exhibit impaired growth, reduced weight, seizures, neurodegeneration, or reduced lifespan.

Also provided are genetically modified mice whose genomes comprise a genetically modified Tor1a locus, further comprising a polynucleotide encoding a Cre recombinase in one or more cells. In some such mice, the mouse exhibits overt dystonia. In some such mice, the mouse does not exhibit impaired growth, reduced weight, seizures, neurodegeneration, or reduced lifespan. Optionally, the polynucleotide is genomically integrated. Optionally, the polynucleotide is extrachromosomal.

In some such mice, the polynucleotide is introduced into the genetically modified mouse via a gene therapy vector. In some such mice, the gene therapy vector is selected from the group consisting of a naked polynucleotide, a polynucleotide complex, and a viral vector. Optionally, the gene therapy vector is a polynucleotide complex, and the polynucleotide complex is a lipid nanoparticle comprising the polynucleotide and lipids. Optionally, the gene therapy vector is a viral vector, and the viral vector is selected from the group consisting of a retrovirus, an adenovirus, a herpes simplex virus, a pox virus, a vaccinia virus, a lentivirus, or an adeno-associated virus (AAV). Further optionally, the viral vector is pseudotyped to a specific tissue. Optionally, the specific tissue is the central and/or peripheral nervous system.

Provided are methods of making a progeny mouse. These methods comprise breeding: I. a first mouse whose genome comprises a genetically modified Tor1a locus, wherein the genetically modified Tor1a locus comprises: (i) a Tor1a allele comprising a ΔGAG deletion associated with DYT1 dystonia in exon 5; and (ii) an inducible Tor1a allele comprising (a) loxP sites flanking a wild type exon 5 of Tor1a and (b) a downstream copy of exon 5 comprising the ΔGAG deletion associated with DYT1 dystonia; with II. a second mouse whose genome comprises a Cre recombinase. The resulting progeny mouse has a genome that comprises the ΔGAG deletion associated with DYT1 dystonia in exon 5 in both alleles of the Tor1a locus in one or more cells or tissues, and the progeny mouse exhibits overt dystonia.

Provided are methods for screening a potential therapeutic agent to treat DYT1 dystonia. The method comprises: I. obtaining the progeny mouse described above; II. administering the potential therapeutic agent to the progeny mouse; and II. assaying one or more behavioral characteristics associated with DYT1 dystonia in the progeny mouse. A decrease in the one or more behavioral characteristics compared to a progeny mouse not receiving the potential therapeutic agent is indicative of an effective therapeutic agent.

Also provided are methods for screening a potential therapeutic agent to treat DYT1 dystonia, comprising: I. obtaining the genetically modified mouse discussed above that further comprises a polynucleotide encoding a Cre recombinase in one or more cells; II. administering the potential therapeutic agent to the genetically modified mouse; and II. assaying one or more behavioral characteristics associated with DYT1 dystonia in the genetically modified mouse. A decrease in the one or more behavioral characteristics compared to a progeny mouse not receiving the potential therapeutic agent is indicative of an effective therapeutic agent.

### BRIEF DESCRIPTION OF THE FIGURES

FIG. 1 shows the breeding scheme used to generate Emx1-Cre^{+/-}Tor1a^{i-ΔE/ΔE} mice. In Cre-expressing cells of the neocortex and hippocampus, two copies of Tor1a with the mutant exon 5 are expressed. Cells that do not express Cre are heterozygous and express one wild type copy of Tor1a and one copy of Tor1a with the mutant exon 5.

FIG. 2 confirms that no unintended effects of the Tor1a i-ΔE allele (also referred to as “Swap” allele) are observed. Mean body weight of various Emx1-Cre-positive Tor1a genotype animals at 7.5 months of age shows no difference across genotypes (bars represent mean±SEM) (Panel A). Western blot analysis of the striatum shows no additional torsinA protein bands in Tor1a^{Swap/+} mice (molecular weight standard in the left lane; units in kD) (Panel B).

FIG. 3 shows functional behavioral characteristics of Emx1-Cre^{+/-}Tor1a^{i-ΔE/ΔE} mice. The beam walking test showed a significant increase in the number of slips over control mice (Panel A). The accelerated rotarod test showed no difference as compared to control mice (latency to fall is in seconds) (Panel B). Quantification of specific abnormal movements associated with overt dystonia in mice during 1 minute of tail suspension showed increased forelimb clasping and truncal twisting compared to control animals (Panel C). In all panels, bars represent mean±SEM and *=p<0.05.

FIG. 4 shows representative images of an overt dystonia phenotype in mice, including forelimb clasping (Panel A), forelimb and hindlimb clasping (Panel B), and truncal twisting (Panel C).

FIG. 5 shows the effect of administration of a common dystonia treatment, trihexyphenidyl (THP), on the overt dystonia phenotype of Emx1-Cre^{+/-}Tor1a^{i-ΔE/ΔE} mice. In the vehicle treatment group, quantification of specific abnormal movements associated with overt dystonia in mice during 1 minute of tail suspension showed increased forelimb clasping and truncal twisting compared to control animals (Panel A). Treatment with THP reduced abnormal dystonia movements to at least the levels observed in control mice (Panel B). Panel C is a direct comparison of the total number of abnormal movements (abnormal movement score) from FIG. 9, Panels A and B, and shows that while abnormal movements in control animals did not vary between groups and were unaffected by THP treatment, abnormal movements in Emx1-Cre^{+/-}Tor1a^{i-ΔE/ΔE} mice were reduced to control levels following THP administration. In all panels, bars represent mean±SEM and *=p<0.05.

FIG. 6 shows the breeding scheme used to generate Rgs9-Cre^{+/-}Tor1a^{i-ΔE/ΔE} mice. In Cre-expressing medium spiny neurons of the striatum, two copies of Tor1a with the mutant exon 5 are expressed. Cells that do not express Cre are heterozygous and express one wild type copy of Tor1a and one copy of Tor1a with the mutant exon 5.

FIG. 7 shows weights and functional behavioral characteristics of Rgs9-Cre^{+/-}Tor1a^{i-ΔE/ΔE} mice. Mean body weight of various Rgs9-Cre-positive Tor1a genotype animals at 6 months of age show no difference across genotypes (Panel A). The beam walking test showed no difference in the number of slips over control mice (Panel B). Similarly, the accelerated rotarod test showed no difference as compared to control mice (latency to fall is in seconds) (Panel C). In all panels, bars represent mean±SEM and *=p<0.05.

FIG. 8 shows a comparison of overt dystonia movements between Rgs9-Cre^{+/-}Tor1a^{i-ΔE/ΔE} mice and Rgs9-Cre-driven conditional torsin-1A knockout mice (CKO mice). Quantification of specific abnormal movements associated with overt dystonia in mice during 1 minute of tail suspension showed increases in all categories for Rgs9-Cre^{+/-}Tor1a^{i-ΔE/ΔE} mice compared to control animals (Panel A). Quantification of specific abnormal movements associated with overt dystonia in mice during 1 minute of tail suspension showed increases in only fore- and hindlimb clasping for CKO mice compared to control animals (Panel B). In all panels, bars represent mean±SEM and *=p<0.05.

FIG. 9 shows the effect of administration of a common dystonia treatment, trihexyphenidyl (THP), on the overt dystonia phenotype of Rgs9-Cre^{+/-}Tor1a^{i-ΔE/ΔE} mice. In the vehicle treatment group, quantification of specific abnormal movements associated with overt dystonia in mice during 1 minute of tail suspension showed increased forelimb clasping and truncal twisting compared to control animals (Panel A). Treatment with THP reduced abnormal dystonia movements to at least the levels observed in control mice (Panel B). Panel C is a direct comparison of the total number of abnormal movements (abnormal movement score) from FIG. 5, Panels A and B, and shows that while abnormal movements in control animals did not vary between groups and were unaffected by THP treatment, abnormal movements in Rgs9-Cre^{+/-}Tor1a^{i-ΔE/ΔE} mice were reduced to control levels following THP administration. In all panels, bars represent mean±SEM and *=p<0.05.

FIG. 10 shows decreased amplitude spontaneous excitatory postsynaptic currents (sEPSCs) in medium spiny neurons (MSNs) of Rgs9-Cre^{+/-}Tor1a^{i-ΔE/ΔE} mice compared to control mice. Panel A shows representative sEPSC traces, and Panel B shows quantification of these measurements. Bars represent mean±SEM and *=p<0.05.

FIG. 11 shows decreased normalized AMPA currents and increased normalized NMDA currents in evoked excitatory postsynaptic currents (eEPSCs) in medium spiny neurons (MSNs) of Rgs9-Cre^{+/-}Tor1a^{i-ΔE/ΔE} mice compared to control mice. Panel A shows representative eEPSC traces (the shaded bar on the trace indicates where NMDA current is measured), and Panel B shows quantification of these measurements. Bars represent mean SEM and *=p<0.05.

### DEFINITIONS

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (−NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

An “open reading frame” or “ORF” is a portion of a DNA which contains a sequence of bases that could potentially encode a protein. As an example, an ORF can be located between the start-code sequence (initiation codon) and the stop-codon sequence (termination codon) of a gene. The term “in frame” refers to coding sequences that are part of the same ORF and could be translated continuously, e.g., as a fusion peptide, propeptide, prepropeptide, etc.

The term “genomically integrated” refers to a nucleic acid that has been introduced into a cell such that the nucleotide sequence is integrated into the genome of the cell and is capable of being inherited by progeny thereof. Any method suitable for stable integration of a nucleic acid into the genome of a cell may be used to form a genomically integrated nucleic acid.

The term “plasmid” or “vector” includes any known vector including a bacterial vector, a viral vector, an episomal plasmid, an integrative plasmid, or a phage vector. The term “vector” refers to a construct which is capable of delivering, and, optionally, expressing, one or more expressible sequences (e.g., protein coding sequence) in a host cell.

The term “targeting vector” refers to a vector that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous” refers to a nucleic acid sequence that occurs naturally within a cell or rodent (e.g., a mouse). For example, an endogenous Tor1a sequence of a rodent refers to a native Tor1a sequence that naturally occurs at the Tor1a locus in the rodent.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two portions that do not naturally occur together in the same molecule. A heterologous sequence can be a sequence which is present in a cell, genome, or gene in the genetic context other than where it naturally occurs. For example, the term “heterologous,” when used with reference to portions of a nucleic acid or portions of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. A heterologous sequence can be a sequence derived from the same gene and/or cell type, but introduced into the cell or a similar cell in a different context, such as on an expression vector or in a different chromosomal location or with a different promoter. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

The term “locus” refers to a specific location of a gene (or significant sequence), DNA sequence, polypeptide-encoding sequence, or position on a chromosome of the genome of an organism. For example, a “Tor1a locus” may refer to the specific location of a Tor1a gene, Tor1a DNA sequence, Tor1a-encoding sequence, or Tor1a position on a chromosome of the genome of an organism that has been identified as to where such a sequence resides. A “Tor1a locus” may comprise a regulatory element of a Tor1a gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Cre recombinase” or “Cre” is a member of the integrase family of recombinases from P1 bacteriophage that catalyzes site-specific recombination of DNA between loxP recognition sites. The loxP site is a 34 base pair sequence including two 13 base pair inverted repeats separated by an 8 base pair spacer. Recombination products depend on the number and orientation of the loxP sites. If two loxP sites are located on different DNA molecules (for example, in trans), translocation between the two molecules (for example, chromosomes) will occur. In other examples, DNA between two loxP sites in the same orientation will be excised and DNA between loxP sites in opposite orientations will be inverted with respect to its starting orientation. See, e.g. Nagy, Genesis 26:99-109, 2000. Cre expression can be operably linked to any promoter of choice as suitable for the desired location and/or timing of Cre expression, and such promoters can be interchanged between species. For example, to obtain global Cre-mediated genetic modification, the Cre gene can be operably linked to an adenoviral EIIa promoter that is expressed very early in mouse development (e.g., in pre-implantation embryos), thus expressing Cre in nearly all tissues of the mouse. Tissue-restricted (or tissue-specific, used interchangeably) promoters, active later in development (i.e., after organ and tissue systems and specific gene expression related thereto has been established), can be employed to restrict Cre-mediated genetic modification to selected tissue, organ, and/or cell types. Additionally, temporal control of Cre activity can be achieved using inducible Cre systems (e.g., CreER^{T2}).

The term “variant” refers to a nucleotide sequence differing from the sequence most prevalent in a population (e.g., by at least one nucleotide) or a protein sequence different from the sequence most prevalent in a population (e.g., by at least one amino acid).

The term “fragment” when referring to a protein means a protein that is shorter or has fewer amino acids than the full-length protein. The term “fragment” when referring to a nucleic acid means a nucleic acid that is shorter or has fewer nucleotides than the full-length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “isolated” with respect to proteins and nucleic acid refers to proteins and nucleic acids that are relatively purified with respect to other bacterial, viral, or cellular components that may normally be present in situ, up to and including a substantially pure preparation of the protein and the polynucleotide. The term “isolated” also includes proteins and nucleic acids that have no naturally occurring counterpart, have been chemically synthesized and are thus substantially uncontaminated by other proteins or nucleic acids, or has been separated or purified from most other cellular components with which they are naturally accompanied (e.g., other cellular proteins, polynucleotides, or cellular components).

The term “in vitro” includes artificial environments and to processes or reactions that occur within an artificial environment (e.g., a test tube). The term “in vivo” includes natural environments (e.g., organism or body) and to processes or reactions that occur within a natural environment. The term “ex vivo” includes cells that have been removed from the body of an individual and to processes or reactions that occur within such cells.

The term “reporter gene” refers to a nucleic acid having a sequence encoding a gene product (e.g., an enzyme or a detectable protein; e.g., a fluorescent protein) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, luciferase genes (e.g., nano luciferase), genes encoding beta-glucuronidase (GUS), and genes encoding fluorescent proteins. A “reporter protein” refers to a protein encoded by a reporter gene.

The term “recombination” includes any process of exchange of genetic information between two polynucleotides and can occur by any mechanism. Recombination in response to double-strand breaks (DSBs) occurs principally through two conserved DNA repair pathways: non-homologous end joining (NHEJ) and homologous recombination (HR). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLOS ONE 7:e45768:1-9; and Wang et al. (2013) Nat Biotechnol. 31:530-532, each of which is herein incorporated by reference in its entirety for all purposes.

As used herein, a “control” as in a control sample or a control subject (e.g., mouse) is a comparator for a measurement, e.g., a diagnostic measurement of a sign or symptom of a disease (e.g., dystonia). In certain aspects, a control can be a subject sample from the same subject an earlier time point, e.g., before a treatment intervention. In certain aspects, a control can be a measurement from a normal subject, i.e., a subject not having the disease of the treated subject, to provide a normal control, e.g., behavioral activity in a subject (e.g., overt dystonia in a mouse). In certain aspects, a control can be an untreated subject with the same disease. In certain aspects, a control can be a subject treated with a different therapy, e.g., the standard of care. In certain aspects, the control is matched for certain factors to the subject being tested, e.g., age, gender. Selection of an appropriate control is within the ability of those of skill in the art.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

In general, the term “about” indicates variation in a quantity of a component of a composition not having a significant effect on the activity or stability of the composition. For example, “about” can mean within 1 standard deviation. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Provided herein are transgenic mouse models of DYT1 dystonia with partially conditional knock-in of the DYT1 dystonia trinucleotide deletion (ΔGAG) in exon 5 of the mouse Tor1a gene (also known as Dyt1). Also provided are methods of generating these mice and methods of their use for preclinical screening of potential dystonia therapeutics.

Current mouse models of DYT1 dystonia fail to adequately recapitulate the phenotype of human DYT1 dystonia, either because they fail to display an overt dystonia phenotype or because they exhibit extraneous phenotypes that are inconsistent with human disease and confound their use in understanding disease pathophysiology and screening potential therapeutic agents. For example, several models display one or more of the following extraneous phenotypes: impaired growth, reduced weight, seizures, neurodegeneration, and reduced lifespan. By contrast, the mouse models provided herein exhibit the overt dystonia phenotype without evidence of impaired growth, alteration in weight, seizures, neurodegeneration, or alteration in life span. These models recapitulate the human DYT1 dystonia phenotype and alteration of this phenotype is more sensitive to known therapeutic agents than existing models, making the provided DYT1 dystonia mouse models superior to existing models; both for the study of DYT1 dystonia pathophysiology and for the screening of potential therapeutic agents.

### II. Torsin Family 1, Member a (Torsin-1A)

Torsin-1A (or torsinA) is a member of the AAA+ superfamily (ATPases associated with diverse cellular activities). Torsin-1A is a protein with chaperone functions important for the control of protein folding, processing, stability, and localization as well as for the reduction of misfolded protein aggregates. It is involved in the regulation of synaptic vesicle recycling and controls STON2 protein stability in collaboration with the COP9 signalosome complex (CSN). In the nucleus, torsin-1A may link the cytoskeleton with the nuclear envelope, and this mechanism seems to be crucial for the control of nuclear polarity, cell movement, and specifically in neurons, nuclear envelope integrity. Torsin-1A also participates in cellular trafficking and may regulate the subcellular location of multi-pass membrane proteins such as the dopamine transporter SLC6A3, leading to modulation of dopamine neurotransmission. In the endoplasmic reticulum, Torsin-1A plays a role in the quality control of protein folding by increasing clearance of misfolded proteins or holding them in an intermediate state for proper refolding.

Deletion of one of a pair of glutamic acids at amino acid positions 302 and 303 (ΔE303) of human torsin-1A (corresponding to amino acid positions 303 and 304 in the mouse ortholog), resulting from a trinucleotide deletion (ΔGAG) in exon 5 of one allele of the TOR1A gene, is the most common cause of DYT1 dystonia in humans. Other mutant protein variants can be involved, however. Biallelic deletion of this glutamic acid residue (or other biallelic mutant variants) results in the far more severe arthrogryposis multiplex congenita 5 (AMC5). Disease pathogenesis in DYT1 dystonia is poorly understood, but it is believed that the ΔE303 variant torsin-1A can exert a dominant negative effect due to its homo-hexameric protein structure, thus reducing the normal activity of wild type torsin-1A produced by the unaffected allele in heterozygous individuals.

Mouse Tor1a maps to 2 B; 2 21.77 cM on chromosome 2 (NCBI RefSeq Gene ID 30931; assembly GRCm39 (GCF_000001635.27); location NC_000068.8 (30850573 . . . 30857930, complement). The wild type mouse torsin-1A protein has been assigned UniProt accession number Q9ER39. The canonical amino acid sequence (NP_659133.1) is set forth in SEQ ID NO: 1. An exemplary mRNA (cDNA) encoding the canonical protein is assigned NCBI Accession No. NM_144884.2 and is set forth in SEQ ID NO: 2. An exemplary coding sequence (CDS) (CCDS15891.1) is set forth in SEQ ID NO: 3. The canonical, full-length mouse torsin-1A protein set forth in SEQ ID NO: 1 has 333 amino acids, including a signal peptide (amino acids 1-20) and the mature protein chain (amino acids 21-333), with N-linked glycosylation modifications occurring at asparagine residues at amino acid positions 144 and 159. Mouse torsin-1A has known regions of interaction with SNAPIN (amino acids 92-252), KLC1 (amino acids 252-333), and SYNE3 (amino acids 313-333). Delineations between these domains and interacting regions are as designated in UniProt. Reference to mouse torsin-1A includes the canonical (wild type) form, as well as all allelic forms and isoforms. Any other forms of mouse torsin-1A have amino acids numbered for maximal alignment with the wild type form, aligned amino acids being designated the same number.

Human TOR1A maps to 9q34.11 on chromosome 9 (NCBI RefSeq Gene ID 1861; assembly GRCh38.p14 (GCF_000001405.40); location NC_000009.12 (129812942 . . . 129824136, complement). The wild type human torsin-1A protein has been assigned UniProt accession number O14656. At least two isoforms are known (O14656-1 and O14656-2, the latter arising from alternative splicing). The sequence for the canonical amino acid isoform (NP_000104.1) is set forth in SEQ ID NO: 4. An exemplary mRNA (cDNA) isoform encoding the canonical protein is assigned NCBI Accession No. NM_000113.3 and is set forth in SEQ ID NO: 5. An exemplary coding sequence (CDS) (CCDS6930.1) is set forth in SEQ ID NO: 6. The canonical, full-length human torsin-1A protein set forth in SEQ ID NO: 4 has 332 amino acids, including a signal peptide (amino acids 1-20) and the mature protein chain (amino acids 21-332), with N-linked glycosylation modifications occurring at asparagine residues at amino acid positions 143 and 158. Mouse torsin-1A has known regions of interaction with SNAPIN (amino acids 91-251), KLC1 (amino acids 251-332), and SYNE3 (amino acids 312-332). Delineations between these domains and interacting regions are as designated in UniProt. Reference to human torsin-1A includes the canonical (wild type) form, as well as all allelic forms and isoforms. Any other forms of human torsin-1A have amino acids numbered for maximal alignment with the wild type form, aligned amino acids being designated the same number.

### II. Partially Conditional ΔGAG Knock-In Mice

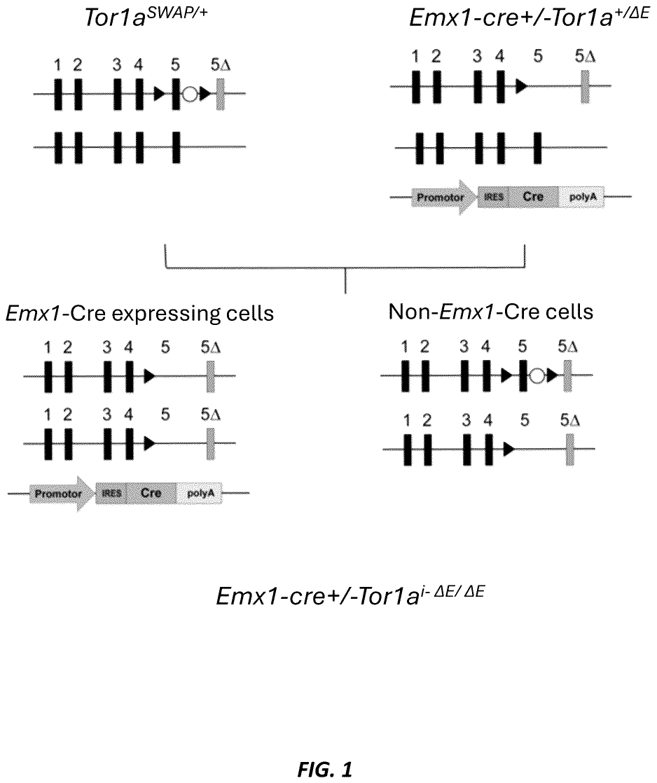

Provided herein are genetically modified mice whose genome comprises a genetically modified Tor1a locus. A diploid organism has two alleles at each genetic locus. Each pair of alleles represents the genotype of a specific genetic locus. Genotypes are described as homozygous if there are two identical alleles at a particular locus and as heterozygous if the two alleles differ. The genetically modified Tor1a locus comprises a first Tor1a allele comprising a ΔGAG deletion associated with DYT1 dystonia in exon 5 (i.e., the ΔE allele) and a second Tor1a allele an inducible allele comprising (a) loxP sites flanking a wild type exon 5 of Tor1a and (b) a downstream copy of exon 5 comprising the ΔGAG deletion associated with DYT1 dystonia (i.e., the i-ΔE allele, also known as a “Swap” allele and referred to interchangeably throughout) (see FIG. 1, bottom right panel). Thus, these genetically modified mice are globally heterozygous for the ΔGAG (ΔE) deletion associated with DYT1 dystonia in exon 5. The second, Cre-inducible ΔE allele expresses wild type torsin-1A in the absence of Cre expression. These genetically modified mice are interchangeably referred to throughout as “partially conditional ΔGAG knock-in mice,” “conditional knock-in mice,” or “Tor1a^{i-ΔE/ΔE} mice.”

The genetically modified mice can be male or female. The genetically modified mice can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 12951/SV, 12951/Svlm), 129S2, 129S4, 129S5, 12959/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/lOScSn, C57BL/l0Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Various methods can be used to generate the targeted genetic modification in the mouse genome of interest. Methods of obtaining the targeted genetic modifications as described in the instant application can comprise unaided homologous recombination, recombinase-based insertion, and DNA repair-based insertion and are known in the art (Dong et al., 2021, PNAS. 118(22) e2004834117). Recombinase-based insertion can comprise systems and constructs involving site-specific recombinases, including but not limited to, Cre:loxP systems, Flp:FRT systems, Dre:rox systems, VCre:loxVsystems, Gin:gix systems, Bxb1:attP attB systems, phiC31:attP attB systems. DNA repair-based insertion methods rely upon the activity of a nuclease agent, including but not limited to a Transcription Activator-Like Effector Nuclease (TALEN; see, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkg704; and Miller et al. (2011) Nature Biotechnology 29:143-148; all of which are herein incorporated by reference), a zinc-finger nuclease (ZFN; see, US20060246567; US20080182332; US20020081614; US20030021776; WO/2002/057308A2; US20130123484; US20100291048; WO/2011/017293A2; and Gaj et al. (2013) Trends in Biotechnology, 31(7):397-405 each of which is herein incorporated by reference), a meganuclease (see, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764 each of which is herein incorporated by reference), or a CRISPR/Cas system (see WO 2013/176772, WO 2014/065596, WO 2014/131833, WO 2016/106121, WO 2019/067910, WO 2020/082042, US 2020/0270617, WO 2020/082041, US 2020/0268906, WO 2020/082046, US 2020/0289628, Cebrian-Serrano and Davies (2017) Mamm. Genome 28(7):247-261, Zetsche et al. (2015) Cell 163(3):759-771, Liu et al. (2019) Nature 566(7743):218-223, and Pausch et al. (2020) Science 369(6501):333-337 each of which is herein incorporated by reference).

Also provided herein are progeny mice obtained by breeding the Tor1a^{i-ΔE/ΔE} mice with a mouse whose genome comprises a Cre recombinase (a “Cre mouse”). In the cells of the progeny mouse that express the Cre recombinase, the wild type copy of exon 5 of Tor1a in the i-ΔE inducible allele is deleted and the downstream copy of exon 5 comprising the ΔGAG deletion is expressed. Like the Tor1a^{i-ΔE/ΔE} mice, the Cre mouse can be male or female, can be of any genetic strain background, and can be produced by any targeted genetic modification technique familiar to those of skill in the art. In certain aspects, the Cre recombinase is expressed under the control of a tissue-specific promoter. Any Cre mouse expressing Cre under the control of a promoter (i.e., operably linked to a promoter) that, when crossed with Tor1a^{i-ΔE/ΔE} mice, results in progeny mice with an overt dystonia phenotype can be used. In certain aspects, the progeny mice do not exhibit impaired growth, reduced weight, seizures, neurodegeneration, or reduced life span.

In a specific example, the genome of the Cre mouse comprises a Cre gene that is operably linked to a mouse Emx1 promoter, thereby restricting Cre-mediated genetic modification to the neocortex. In a certain aspect, the resulting progeny mice (e.g., Emx1-Cre^{+/-}Tor1a^{i-ΔEΔE}) exhibit overt dystonia. In further aspect, the resulting progeny mice (e.g., Emx1-Cre^{+/-}Tor1a^{i-ΔE/ΔE}) do not exhibit impaired growth, reduced weight, seizures, neurodegeneration, or reduced life span. In another example, the genome of the Cre mouse comprises a Cre gene that is operably linked to a mouse Rgs9 promoter, thereby restricting Cre-mediated genetic modification to the striatum medium spiny neurons. In a certain aspect, the resulting progeny mice (e.g., Rgs9-Cre^{+/-}Tor1a^{i-ΔE/ΔE}) exhibit overt dystonia. In a further aspect, the resulting progeny mice (e.g., Rgs9-Cre^{+/-}Tor1a^{i-ΔE/ΔE}) do not exhibit impaired growth, reduced weight, seizures, neurodegeneration, or reduced life span.

Also provided herein are Tor1a^{i-ΔE/ΔE} mice further comprising a polynucleotide encoding a Cre recombinase in one or more cells. In a certain aspect, these mice exhibit overt dystonia. In a further aspect, these mice do not exhibit impaired growth, reduced weight, seizures, neurodegeneration, or reduced life span. The polynucleotide encoding the Cre recombinase can be DNA or RNA. The polynucleotide encoding the Cre recombinase can be genomically integrated or extrachromosomal (e.g., as an episome). Methods for introduction of the polynucleotide into the one or more cells of the Tor1a^{i-ΔE/ΔE} mice are provided elsewhere herein.

### IV. Methods of Generating the Dystonia Mouse Models

A. Breeding with Cre-Expressing Mice

Those skilled in the art will recognize that numerous animal breeding strategies can be devised to obtain a particular genetically modified mouse, any of which will ultimately result in the desired genotype, albeit with varying efficiency. The breeding strategies described below and elsewhere herein are merely exemplary, and any effective breeding strategy can be used to obtain the Emx1-Cre^{+/-}Tor1a^{i-ΔE/ΔE} and/or Rgs9-Cre^{+/-}Tor1da^{i-ΔE/ΔE} dystonia models provided herein and described in detail above. The basic principles to formulate breeding strategies to obtain particular genotypes in transgenic animals, based on Mendelian inheritance, are familiar to those of skill in the art. Any of the mice described in the provided methods can be male or female, can be of any genetic strain background, and can be produced by any targeted genetic modification technique familiar to those of skill in the art.

Provided are methods of making a progeny mouse that exhibits overt dystonia. In certain aspects, the progeny mouse does not exhibit impaired growth, reduced weight, seizures, neurodegeneration, or reduced life span. The method comprises breeding a first mouse whose genome comprises a genetically modified Tor1a locus, wherein the genetically modified Tor1a locus comprises: (i) a Tor1a allele comprising a ΔGAG deletion associated with DYT1 dystonia in exon 5 (i.e., the ΔE allele); and (ii) an inducible Tor1a allele comprising (a) loxP sites flanking a wild type exon 5 of Tor1a and (b) a downstream copy of exon 5 comprising the ΔGAG deletion associated with DYT1 dystonia (i.e., the i-ΔE allele); with a second mouse whose genome comprises a Cre recombinase (a “Cre mouse”).

In a specific example, Emx1-Cre^{+/-}Tor1a^{i-ΔE/ΔE} mice are generated by breeding Tor1a^{i-ΔE/+} mice (also known as Tor1a^{Swap/+} mice) with Emx1-Cre^{+/-}Tor1a^{+/ΔE} mice (see FIG. 1). This cross results in the production of eight possible genotypes of progeny, as follows: (1) Emx1-Cre^{+/-}Tor1a^{i-ΔE/+} mice; (2) Emx1-Cre^{-/-}Tor1a^{i-ΔE/+} mice; (3) Emnx1-Cre^{+/-}Tor1a^{i-ΔE/ΔE} mice; (4) Emx1-Cre^{-/-}Tor1a^{i-ΔE/ΔE} mice; (5) Emx1-Cre^{+/-}Tor1a^{+/+} mice; (6) Emx1-Cre^{-/-}Tor1a^{+/+} mice; (7) Emx1-Cre^{+/-}Tor1a^{+/ΔE} mice; and (8) Emx1-Cre^{-/-}Tor1a^{+/ΔE} mice.

In another example, Rgs9-Cre^{+/-}Tor1a^{i-ΔE/ΔE} mice are generated by breeding Tor1a^{i-ΔE/+} mice (also known as Tor1a^{Swap/+} mice) with Rgs9-Cre^{+/-}Tor1a^{+/ΔE} mice (see FIG. 1). This cross results in the production of eight possible genotypes of progeny, as follows: (1) Rgs9-Cre^{+/-}Tor1a^{i-ΔE/+} mice; (2) Rgs9-Cre^{-/-}Tor1a^{i-ΔE/+} mice; (3) Rgs9-Cre^{+/-}Tor1a^{i-ΔE/ΔE} mice; (4) Rgs9-Cre^{-/-}Tor1a^{i-ΔE/ΔE} mice; (5) Rgs9-Cre^{+/-}Tor1a^{+/+}mice; (6) Rgs9-Cre^{-/-}Tor1a^{+/+} mice; (7) Rgs9-Cre^{+/-}Tor1a^{+/ΔE} mice; and (8) Rgs9-Cre^{-/-}Tor1a^{+/ΔE} mice.

B. Introduction of a Polynucleotide Encoding Cre into Tor1a^{i-ΔE/ΔE} Mice

Also provided are methods for generating a mouse that exhibits overt dystonia by introducing a polynucleotide encoding a Cre recombinase into one or more cells of a Tor1a^{i-ΔE/ΔE} mouse. In a certain aspect, such a mouse does not exhibit impaired growth, reduced weight, seizures, neurodegeneration, or reduced lifespan. In a certain aspect, the polynucleotide is introduced into the mouse in a gene therapy vector. A vector can comprise additional sequences such as, for example, replication origins, promoters, and genes encoding antibiotic resistance.

Some vectors may be circular. Alternatively, the vector may be linear. The vector can be a naked polynucleotide. The vector can be packaged for delivery via a polynucleotide complex, such as, for example, a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In one aspect, the vector can be a lipid nanoparticle (LNP) comprising the polynucleotide encoding a Cre recombinase. The lipid nanoparticle can, however, comprise the Cre recombinase in any form (e.g., protein, DNA, or RNA). The polynucleotide can be modified to comprise one or more stabilizing modifications familiar in the art. Delivery through such methods can result in transient Cre expression, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes.

In another aspect, the vector can be a viral vector. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cre). A viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors to deliver transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In rAAV genomes for transgene delivery (e.g., Cre), a gene expression cassette is placed between ITR sequences. Typically, rAAV genome cassettes comprise of a promoter to drive expression of a transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther Methods Clin. Dev. 8:87-104, herein incorporated by reference in its entirety for all purposes.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding a heterologous polypeptide (e.g., Cre). The AAV vector may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for brain include AAV1, AAV2, AAV5, AAV7, AAV8, and AAV9, all of which show strong preference for neurons. Additional details regarding AAV vectors in neuroscience applications are reviewed in Haggerty et al. (2020) Mol. Ther Methods Clin. Dev. 17:69-82, herein incorporated in its entirety for all purposes.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V Other pseudotyped/modified AAV variants include AAV2/1, AAV2/6, AAV2/7, AAV2/8, AAV2/9, AAV2.5, AAV8.2, and AAV/SASTG.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene expression, rAAV-delivered rAAV episomes provide long-term, promoter-driven gene expression in non-dividing cells. However, this rAAV-delivered episomal DNA is diluted out as cells divide.

Administration of any of the gene therapy vectors discussed above to introduce the polynucleotide encoding Cre recombinase into the Tor1a^{i-ΔE/ΔE} mouse can be accomplished by any suitable route and means, as appropriate for the vector. For example, administration (i.e., introduction of the polynucleotide) in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes.

The dystonia mouse models provided, generated by any means described elsewhere herein, can be used to screen potential therapeutic agents for the treatment of DYT1 dystonia. In certain aspects, progeny mice are generated from breeding the partially conditional ΔGAG knock-in mice with Cre-expressing mice, by any means as described above, for use in screening potential dystonia therapeutic agents. In other aspects, a polynucleotide encoding Cre (or Cre protein) is introduced into the partially conditional ΔGAG knock-in mice, by any means described above, to generate mice for use in screening potential dystonia therapeutic agents. In yet further aspects, Emx1-Cre^{+/-}Tor1a^{i-ΔE/ΔE} mice and/or Rgs9-Cre^{+/-}Tor1a^{i-ΔE/ΔE} mice are obtained (e.g., commercially, etc.) for use in screening potential dystonia therapeutic agents. In all cases, screening of a potential dystonia therapeutic agent using the dystonia mouse models provided comprises administering the potential therapeutic agent to the mouse and assaying one or more behavioral characteristics associated with DYT1 dystonia in the progeny mouse. The potential therapeutic agent can be compared to a vehicle control, to a known therapeutic agent (e.g., THP), to other potential therapeutic agents, to the same therapeutic agent via alternative administration routes, or any combination thereof. In certain aspects, a decrease in the one or more behaviors compared to a progeny mouse not receiving the potential therapeutic agent is indicative of an effective therapeutic agent.

### V. Methods of Screening Potential Therapeutic Agents Using the Dystonia Mouse Models

The dystonia mouse models provided, generated by any means described elsewhere herein, can be used to screen potential therapeutic agents for the treatment of DYT1 dystonia. In certain aspects, progeny mice are generated from breeding the partially conditional ΔGAG knock-in mice with Cre-expressing mice, by any means as described above, for use in screening potential dystonia therapeutic agents. In other aspects, a polynucleotide encoding Cre (or Cre protein) is introduced into the partially conditional ΔGAG knock-in mice, by any means described above, to generate mice for use in screening potential dystonia therapeutic agents. In yet further aspects, Emx1-Cre^{+/-}Tor1a^{i-ΔE/ΔE} mice and/or Rgs9-Cre^{+/-}Tor1a^{i-ΔE/ΔE} mice are obtained (e.g., commercially, etc.) for use in screening potential dystonia therapeutic agents. Mice can be any relevant postnatal age in which assessing the activity of potential dystonia therapeutic agent is desired.

In all cases, screening of a potential dystonia therapeutic agent using the dystonia mouse models provided comprises administering the potential therapeutic agent to the mouse and assaying one or more behavioral characteristics associated with DYT1 dystonia in the progeny mouse. The potential therapeutic agent can be compared to a vehicle control, a known therapeutic agent (e.g., THP), other potential therapeutic agents, the same therapeutic agent via alternative administration routes or delivery methods, or any combination thereof. In certain aspects, a decrease in the one or more behaviors compared to a progeny mouse not receiving the potential therapeutic agent is indicative of an effective therapeutic agent.

The potential dystonia therapeutic agent (“agent”) can be of any type, such as, for example, nucleic acids, proteins, nucleic-acid-protein complexes, peptide mimetics, antigen-binding proteins, small molecules, or any combination thereof “Administering” or “introducing” includes presenting to the mouse the agent in such a manner that the agent gains access to the interior of cells within the mouse. The introducing can be accomplished by any means suitable for introducing the agent into a cell. If multiple agents are introduced, they can be introduced simultaneously or sequentially, in any combination. In addition, two or more agents can be introduced into a mouse by the same delivery method or different delivery methods. Similarly, two or more agents can be introduced into a mouse by the same route of administration or different routes of administration.

Administration (i.e., introduction of the agent) in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the agent (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of the agent are administered systemically.

Potential therapeutic agents for treating dystonia can be formulated using one or more physiologically and pharmaceutically acceptable carriers, diluents, excipients, or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the agent and the route of administration, among other factors. The introduction of such agents into the mouse can be performed one time or multiple times over a period of time. Intervals may be regular or irregular based on response in the rodent, requirements for dosing of the agent being tested, or other factors.

Any suitable method can be used for assaying one or more behavioral characteristics associated with DYT1 dystonia to determine if the agent being screened is effective in decreasing these behaviors. Such behavioral assays in mice related to motor conditions are familiar to those of skill in the art, including but not limited to, the tail suspension test, cylinder test, beam walking test, rotarod test, adhesive removal test, prepulse inhibition, open field analysis, footprint analysis, and other various scoring systems of abnormal involuntary movements. Scoring and counting of abnormal involuntary movements can be performed manually by an individual (e.g., an investigator observing the animal move and noting the number of abnormal movements) or automated using computerized systems (e.g., videographic analysis, laser beam breaks in open field, etc.). In a certain aspect, the assaying comprises a beam walking test, rotarod test, and/or tail suspension test. In another aspect, the assaying comprises a tail suspension test analyzed using DeepLabCut (DLC) markerless pose estimation of user-defined body parts with deep learning as described in Berryman et al. (2023) Behav. Brain Res. 439:114221, which is hereby incorporated by reference in its entirety for all purposes. Assaying the one or more behavioral characteristics associated with DYT1 dystonia can also comprise electrophysiological studies to analyze motor function, such as, for example, burst duration, burst frequency, co-contractions, and oscillatory activity. An observed decrease in the one or more behavioral characteristics compared to a mouse not receiving the agent is considered to be indicative of an effective therapeutic agent.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website, or the like are published at different times, the version most recently published at the effective filing date of the application is meant, unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases and one letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 1
Description of Sequences

| SEQ ID NO | Type | Description |
|---|---|---|
| 1 | Protein | Mouse torsin-1A protein |
| 2 | DNA | Mouse Tor1a cDNA |
| 3 | DNA | Mouse Tor1a CDS |
| 4 | Protein | Human torsin-1A protein |
| 5 | DNA | Human TOR1A cDNA |
| 6 | DNA | Human TOR1A CDS |

### EXAMPLES

### Example 1. Generation and Characterization of Enx1-Cre^{+/-}Tor1a^{i-ΔE/ΔE} Mice Animals

A DYT1 dystonia conditional knock-in mouse model, Tor1a^{i-ΔE/ΔE}, was developed using Cre-loxP technology and Swap mutant mice. Swap mutant mice possess loxP sites that flank the exon 5 of the Tor1a (alternatively known as Dyt1) gene and an additional downstream mutant exon 5 containing a ΔGAG knock-in mutation. When combined with a tissue-specific Cre recombinase, the wild type exon 5 is deleted, and the ΔGAG knock-in will be expressed (i-ΔE). We generated and characterized mice in which the conditional Tor1a^{i-ΔE/ΔE} knock-in line was crossed with neocortex-specific Emx1-Cre mice. This mouse line is in a heterozygous Tor1a ΔGAG knock-in background (ΔE). Animals were generated following the breeding scheme of Emx1-Cre^{+/-}-Tor1a^{+/ΔE} males with Tor1a^{Swap/+} females to generate Emx1-Cre^{+/-}-Tor1a^{i-ΔE/ΔE} (FIG. 1). The mice were born in a Mendelian ratio. No premature lethality was observed in the mutant mice.

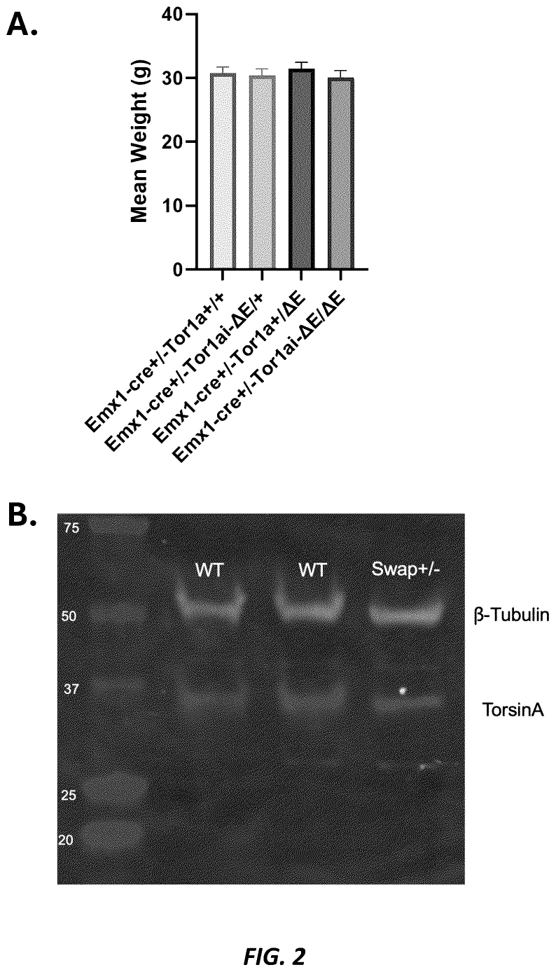

Body weight was analyzed using the SAS GENMOD procedure with normal distribution. There was no significant difference between the body weight of Emx1-Cre^{+/-}Tor1a^{i-ΔE/ΔE}, Emx1-Cre^{+/-}-Tor1a^{+/+}, Emx1-Cre^{+/-}Tor1a^{i-ΔE/+}, and Emx1-Cre^{+/-}Tor1a^{+/ΔE} at approximately 7.5 months age (FIG. 2; Panel A). No sex differences were observed in weight between genotypes (not shown).

### Western Blot

Western blot analysis was conducted to confirm that the presence of multiple exon 5 copies in the Swap allele did not cause alternative splicing events that may have introduced a novel torsin-1A protein. For western blot analysis, 2 males and 2 females each of Tor1a^{+/+} and Tor1a^{Swap/+} age-matched adult mice were sacrificed. After sacrifice, the striatum was dissected and quickly frozen in liquid nitrogen. The striata were homogenized in 200 μL of ice-cold lysis buffer [50 mM Tris-Cl (pH 7.4), 175 mM NaCl, 5 mM EDTA, protease inhibitor tablets (Roche Ref: 04693124001)]and sonicated for 30 seconds to generate cell lysates. Triton X-100 was added to cell lysates to a final concentration of 1%, vortexed, and incubated for 30 minutes on ice. These lysates were centrifuged at 10,000 g for 15 minutes at 4° C. to collect the supernatant. A Bradford assay was performed using bovine serum albumin as standard to calculate protein concentration, which was subsequently diluted to 1 μg/μl for loading. The samples were mixed with SDS-PAGE loading buffer, boiled for 5 minutes, chilled on ice for 1 minute, and then centrifuged for 5 minutes to obtain the supernatant. 15 μg of each sample was then loaded into 10% Tris-Glycine SDS gels. The separated protein bands were transferred to Millipore Immobilon-FL transfer polyvinylidene difluoride (PVDF) membrane at 100 V for 1 hour. After blocking, the membrane was incubated overnight at 4° C. with a mouse primary β-tubulin antibody for internal loading control and a rabbit primary torsinA antibody (Ab34540: Abcam). The membrane was then incubated with anti-rabbit and anti-goat secondary antibodies (LI-COR) for an hour at room temperature, blocked from light. Membranes were imaged using an Odyssey CLx-imager to visualize torsinA and the β-tubulin standard control. Molecular weight was estimated by comparing the migration distance of the corresponding protein band with the pre-stained protein standard ladder (Bio-rad, #1610373).

Western blot analysis of the striatum shows no additional torsinA protein bands in Tor1a^{Swap/+} mice (FIG. 2; Panel B).

### Beam-Walking

The beam-walking test was used to assess the ability of mice to maintain motor coordination and balance. Mice were placed in the sound-attenuated testing room for 1 hour before testing to acclimate prior to testing on each of the four test days. Initially, mice were trained to traverse a medium square beam (14 mm wide) in three consecutive trials, each day, for the first two days. On the third day, the mice were tested in two trials on the medium square beam and then in two trials on the medium round beam (17 mm diameter). On the fourth day, the mice were first tested in two trials on a small round beam (10 mm diameter) and then in two trials on a small square beam (7 mm wide). All beams were 100 cm long. Investigators counted the number of hindlimb slips on each side, blind to the genotypes. Hindlimb slips were counted in the middle 80 cm of the beam. Increased slips indicate a motor deficit. The beam-walking cohort included 25 control mice (16 females and 9 males) and 11 mutant mice (7 females and 4 males). The average age was approximately 8 months, ranging from 3 to 10 months.

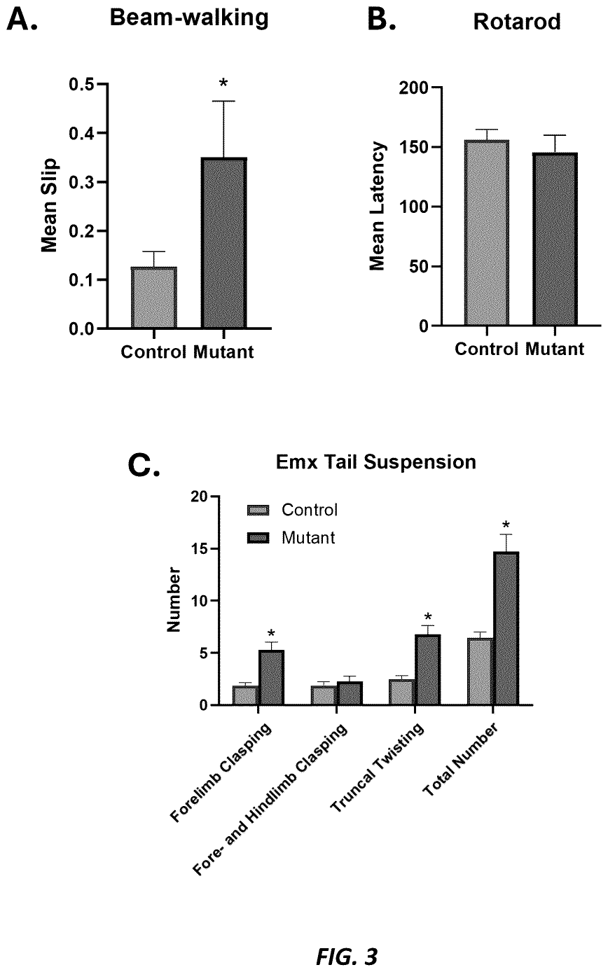

The beam-walking test showed significantly increased slips for the mutant group (n=11, mean slips 0.35±0.11) compared to the control group (n=25, mean slips 0.13±0.03) with a p-value of 0.015 (FIG. 3; Panel A). There were no sex differences.

### Accelerated Rotarod

The accelerated rotarod test was used as another means to assess the ability of mice to maintain coordination and balance. Mice were placed in the sound-attenuated testing room for 1 hour before testing to acclimate. The apparatus (Ugo Basile) started at an initial speed of 4 rpm and accelerated at a rate of 0.2 rpm/s with a final rate of 64 rpm after 5 min. Mice were tested on two consecutive days with three trials per day. Each trial within a day was performed approximately 1 hour apart. The latency to fall was measured with a cutoff time of 5 min. Latency to fall measures motor deficits, with a decreased latency to fall indicative of a motor deficit in the animal. The rotarod cohort included 25 control mice (16 females and 9 males) and 11 mutant mice (7 females and 4 males). The average age was approximately 8 months, ranging from 3 to 10 months.

The accelerated rotarod test showed no significant difference in the performance of the mutant group (Emx1-Cre^{+/-}Tor1a^{i-ΔE/ΔE}) and the control group (Emx1-Cre^{+/-}Tor1a^{i-ΔE/+} and Emx1-Cre^{+/-}Tor1a^{+/ΔE}) with a p-value of 0.53 (FIG. 3; Panel B). There were no sex differences.

### Tail Suspension

The tail suspension test was performed to assess abnormal postural behavior. Before testing, mice were placed in the sound-attenuated testing room for 1 hour to acclimate. Mice were picked up by the tail, suspended in the air for 60 seconds, 6 inches above the ground, and videotaped. The number of forelimb clasping, forelimb and hindlimb clasping, and truncal twisting events were quantified. Each mouse was given an overall score based on the total number of forelimb clasping, forelimb and hindlimb clasping, and truncal twisting events (i.e., an abnormal movement score). The tail suspension cohort included 22 control mice (14 females and 8 males) and 11 mutant mice (7 females and 4 males). The average age was approximately 12 months, ranging from 9 to 20 months.

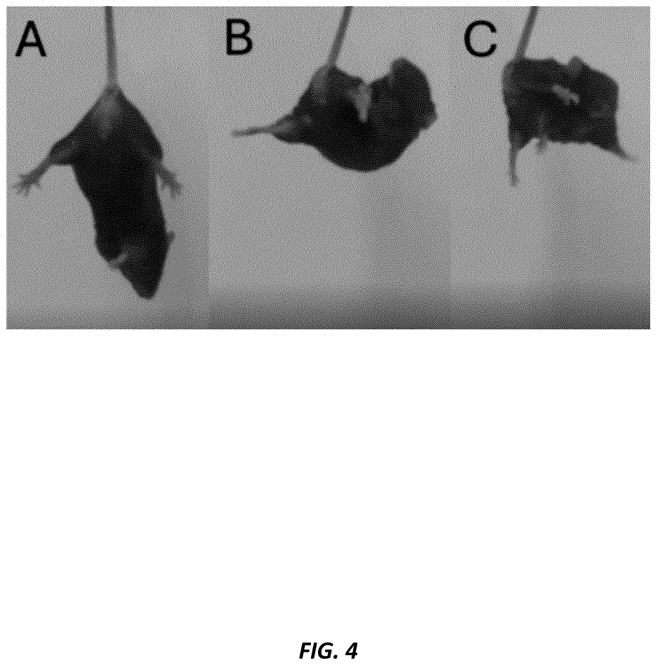

Overt dystonia in the mice was defined as forelimb clasping, forelimb and hindlimb clasping, and/or truncal twisting. Tail suspension results showed a significant overt dystonia phenotype in the mutant group compared to the control group. The mutant group (Emx1-Cre^{+/-}Tor1a^{i-ΔE/ΔE}) showed significantly increased forelimb clasping (mutant, n=11: 5.78±0.78; control, n=22: 1.85±0.30) and truncal twisting (mutant: 6.81±0.82; control: 2.48±0.34) compared to the control group, with a p-value of <0.0001 (FIG. 3; Panel C). Combined forelimb and hindlimb clasping between the mutant and control groups were not significantly different, with a p-value of 0.50 (FIG. 3; Panel C). Images representative of each of these behaviors in mice are provided in FIG. 4, Panels A-C.

### Trihexyphenidyl Treatment

Trihexyphenidyl is a common anticholinergic treatment for DYT1 dystonia. Trihexyphenidyl was given to observe a possible relief of overt dystonia symptoms. Mutant and control animals were treated with either a vehicle saline solution or trihexyphenidyl injected intraperitoneally, daily, for three days at 0.8 mg/kg. Out of 43 total mice, 24 mice received trihexyphenidyl treatment (4 mutant females, 2 mutant males, 11 control females, and 7 control males) and 19 received vehicle treatment (3 mutant females, 2 mutant males, 10 control females, and 4 control males). Approximately an hour after treatment on the third day, the mice underwent the tail suspension test, as described previously. The number of abnormal movements between the vehicle and trihexyphenidyl groups was compared.

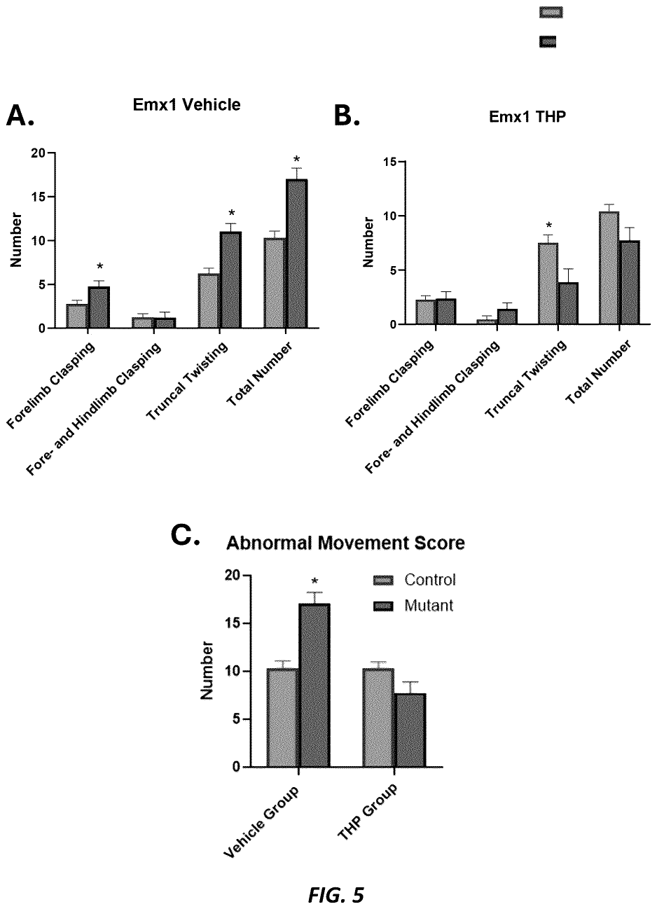

In the vehicle treatment group, quantification of specific abnormal movements associated with overt dystonia in mice during 1 minute of tail suspension showed increased forelimb clasping and truncal twisting compared to control animals (mutant, n=5: 17.05±1.20; control, n=19: 10.28±0.81), with a p-value of <0.0001 (FIG. 5; Panel A). Treatment with THP reduced abnormal dystonia movements to at least the levels observed in control mice, with a p-value of 0.52 (FIG. 5; Panel B). FIG. 5, Panel C is a direct comparison of the total number of abnormal movements (abnormal movement score) from FIG. 5, Panels A and B, and shows that while abnormal movements in control animals did not vary between groups and were unaffected by THP treatment, abnormal movements in Emx1-Cre^{+/-}Tora^{i-ΔE/ΔE} mice were reduced to control levels following THP administration.

### Statistics

Statistical analysis was conducted using SAS software. Distribution was assessed using JMP Pro 17. The rotarod data, beam-walking, and tail suspension data were analyzed using SAS GENMOD and GEE procedures. Significance was assigned at p<0.05.

### Example 2. Generation and Characterization of Rgs9-Cre^{+/-}Tora^{i-ΔE/ΔE} Mice

### Animals

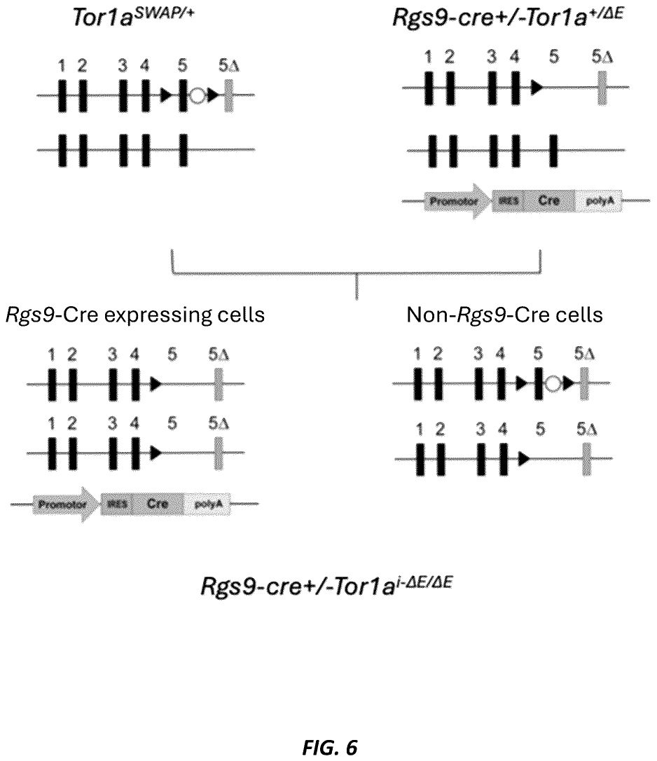

A DYT1 dystonia conditional knock-in mouse model, Tor1a^{i-ΔE/ΔE}, was developed using Cre-loxP technology and Swap mutant mice. Swap mutant mice possess loxP sites that flank the exon 5 of the Tor1a (alternatively known as Dyt1) gene and an additional downstream mutant exon 5 containing a ΔGAG knock-in mutation. When combined with a tissue-specific Cre recombinase, the wild type exon 5 is deleted, and the ΔGAG knock-in will be expressed (i-ΔE). We generated and characterized mice in which the conditional Tor1a^{i-ΔE/ΔE} knock-in line was crossed with striatum-specific Rgs9-Cre mice. This mouse line is in a heterozygous Tor1a ΔGAG knock-in background (ΔE). Animals were generated following the breeding scheme of Rgs9-Cre^{+/-}-Tor1a^{+/ΔE} males with Tor1a^{Swap/+} or Tor1a^{Swap/Swap} females to generate Rgs9-Cre^{+/-}Tor1a^{i-ΔE/ΔE} (FIG. 6). The mice were born in a Mendelian ratio. No premature lethality was observed in the mutant mice.

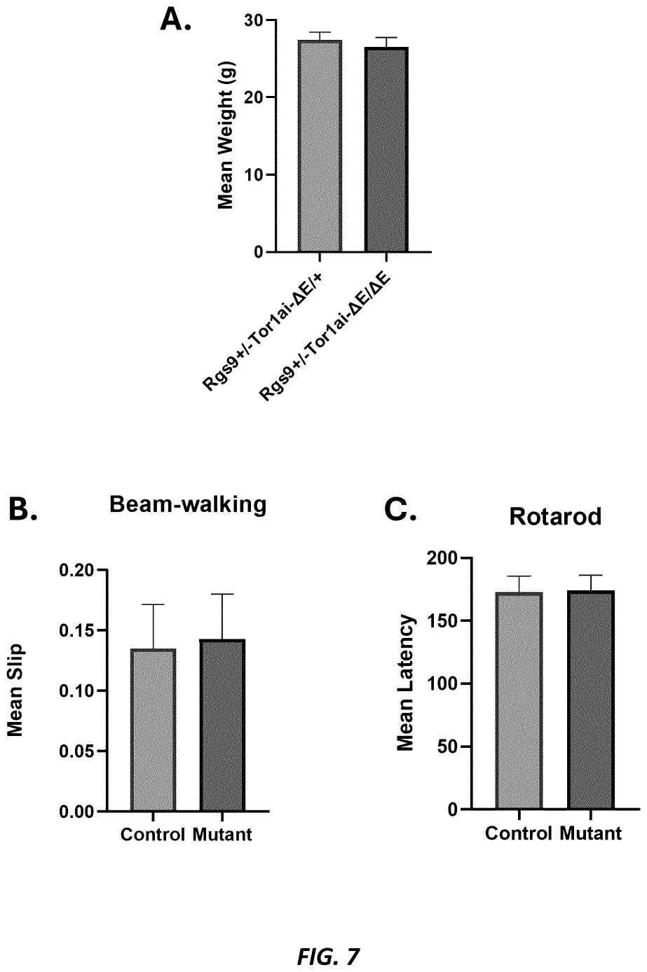

Body weight was analyzed using the SAS GENMOD procedure with normal distribution. There was no significant difference between the body weight of Rgs9-Cre^{+/-}Tor1a^{i-ΔE/ΔE} and Rgs9-Cre^{+/-}Tor1a^{i-ΔE/+} mice at approximately six months of age (FIG. 7; Panel A). No sex differences were observed in weight between genotypes (not shown).

### Beam-Walking

The beam-walking test was used to assess the ability of mice to maintain motor coordination and balance. Mice were placed in the sound-attenuated testing room for 1 hour before testing to acclimate prior to testing on each of the four test days. Initially, mice were trained to traverse a medium square beam (14 mm wide) in three consecutive trials, each day, for the first two days. On the third day, the mice were tested in two trials on the medium square beam and then in two trials on the medium round beam (17 mm diameter). On the fourth day, the mice were first tested in two trials on a small round beam (10 mm diameter) and then in two trials on a small square beam (7 mm wide). All beams were 100 cm long. Investigators counted the number of hindlimb slips on each side, blind to the genotypes. Hindlimb slips were counted in the middle 80 cm of the beam. Increased slips indicate a motor deficit. The beam-walking cohort included 25 control mice (16 females and 9 males) and 11 mutant mice (7 females and 4 males). The average age was approximately 4 months, ranging from 2 to 10 months.

The beam-walking test showed no difference in the mutant group (Rgs9-Cre^{+/-}Tor1a^{i-ΔE/ΔE}) compared to the control group (Rgs9-Cre^{+/-}Tor1a^{i-ΔE-+}), with a p-value of 0.86 (FIG. 7; Panel B). There were no sex differences.

### Accelerated Rotarod

The accelerated rotarod test was used as another means to assess the ability of mice to maintain coordination and balance. Mice were placed in the sound-attenuated testing room for 1 hour before testing to acclimate. The apparatus (Ugo Basile) started at an initial speed of 4 rpm and accelerated at a rate of 0.2 rpm/s with a final rate of 64 rpm after 5 min. Mice were tested on two consecutive days with three trials per day. Each trial within a day was performed approximately 1 hour apart. The latency to fall was measured with a cutoff time of 5 min. Latency to fall measures motor deficits, with a decreased latency to fall indicative of a motor deficit in the animal. The rotarod cohort included 25 control mice (16 females and 9 males) and 11 mutant mice (7 females and 4 males). The average age was approximately 4 months, ranging from 2 to 10 months.

The accelerated rotarod test showed no difference in the mutant group (Rgs9-Cre^{+/-}Tor1a^{i-ΔE/ΔE}) compared to the control group (Rgs9-Cre^{+/-}Tor1a^{i-ΔE/+}), with a p-value of 0.93 (FIG. 7; Panel C). There were no sex differences.

### Tail Suspension

The tail suspension test was performed to assess abnormal postural behavior. Before testing, mice were placed in the sound-attenuated testing room for 1 hour to acclimate. Mice were picked up by the tail, suspended in the air for 60 seconds, 6 inches above the ground, and videotaped. The number of forelimb clasping, forelimb and hindlimb clasping, and truncal twisting events were quantified. Each mouse was given an overall score based on the total number of forelimb clasping, forelimb and hindlimb clasping, and truncal twisting events (i.e., an abnormal movement score). The tail suspension cohort included 9 control mice (4 females and 5 males) and 8 mutant mice (2 females and 6 males). The average age was approximately 6 months, ranging from 4 months to 11 months.

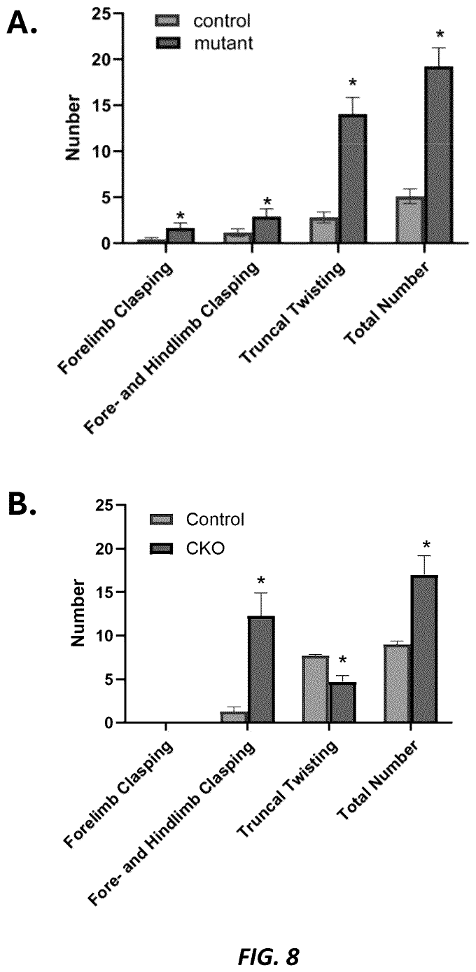

Overt dystonia in the mice was defined as forelimb clasping, forelimb and hindlimb clasping, and/or truncal twisting. Tail suspension results showed a significant overt dystonia phenotype in the mutant group (Rgs9-Cre^{+/-}Tor1a^{i-ΔE/ΔE}) compared to the control group (Rgs9-Cre^{+/-}Tor1a^{i-ΔE/+}). Forelimb clasping was significantly increased for the mutant group (1.66±0.54) compared to the control group (0.40±0.22), with a p-value of 0.03, n=17. Combined forelimb and hindlimb clasping for the mutant group was significantly increased (2.88±0.86) compared to the control group (1.15±0.43), with a p-value of 0.0333. The mutant group also showed significantly more truncal twisting (14.04±1.82) compared to the control group (2.81±0.60), with a p-value of <0.0001 (FIG. 8; Panel A). Images representative of each of these behaviors in mice are provided in FIG. 4, Panels A-C.

To further test whether the torsin-1A ΔE mutation is a loss of function mutation or a toxic gain of function mutation, the conditional knock-in mice were compared to the corresponding conditional knockout animal (CKO; Rgs9-Cre^{+/-}Tor1a^{loxP/Δ}) in tail suspension tests at approximately 6.5 months of age. The CKO mice showed significantly more total dystonic behavior (17.03±2.16) than their respective controls (9.00±0.43), with a p-value of <0.0001, n=12 (FIG. 8; Panel B). By contrast to the conditional knock-in mice, which exhibited overt dystonic movements across all categories, the total score for CKO mice was driven mainly by forelimb and hindlimb clasping, suggesting differences in mechanism between conditional knock-in and CKO mice.

### Trihexyphenidyl Treatment

Trihexyphenidyl is a common anticholinergic treatment for DYT1 dystonia. Trihexyphenidyl was given to observe a possible relief of overt dystonia symptoms. Mutant and control animals were treated with either a vehicle saline solution or trihexyphenidyl injected intraperitoneally, daily, for two days at 0.8 mg/kg. Out of 18 total mice, 10 mice received trihexyphenidyl treatment (1 mutant female, 3 mutant males, 3 control females, and 3 control males) and 8 received vehicle treatment (1 mutant female, 2 mutant males, 2 control females, and 3 control male). Approximately an hour after treatment on the second day, the mice underwent the tail suspension test, as described previously. The number of abnormal movements between the vehicle and trihexyphenidyl groups was compared.

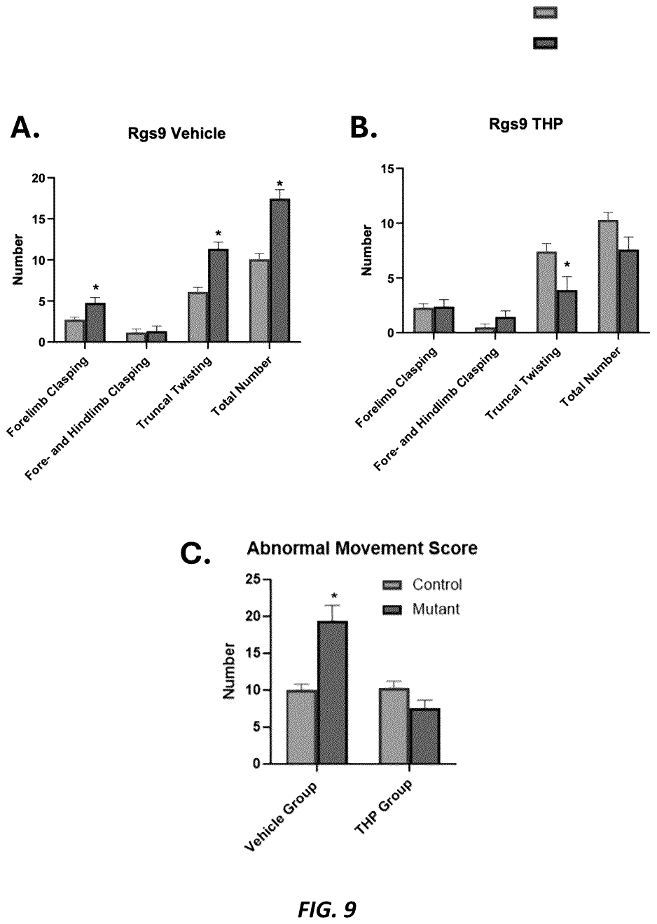

In the vehicle treatment group, quantification of specific abnormal movements associated with overt dystonia in mice during 1 minute of tail suspension showed increased forelimb clasping and truncal twisting compared to control animals (mutant, 17.4636±1.08; control, 10.07±0.73), with a p-value of <0.0001, n=8 (FIG. 9; Panel A). Treatment with THP reduced abnormal dystonia movements to at least the levels observed in control mice, with a p-value of 0.14 (FIG. 9; Panel B). FIG. 9, Panel C is a direct comparison of the total number of abnormal movements (abnormal movement score) from FIG. 9, Panels A and B, and shows that while abnormal movements in control animals did not vary between groups and were unaffected by THP treatment, abnormal movements in Rgs9-Cre^{+/-}Tor1a^{i-ΔE/ΔE} mice were reduced to control levels following THP administration.

### Statistics

Statistical analysis was conducted using SAS software. Distribution was analyzed using JMP Pro 17. The rotarod data, beam-walking, and tail suspension data were analyzed using SAS GENMOD and GEE procedures. Significance was assigned at p<0.05.

### Example 3. Medium Spiny Neuron Electrophysiology in Rgs9-Cre^{+/-}Tora^{i-ΔE/ΔE} Mice

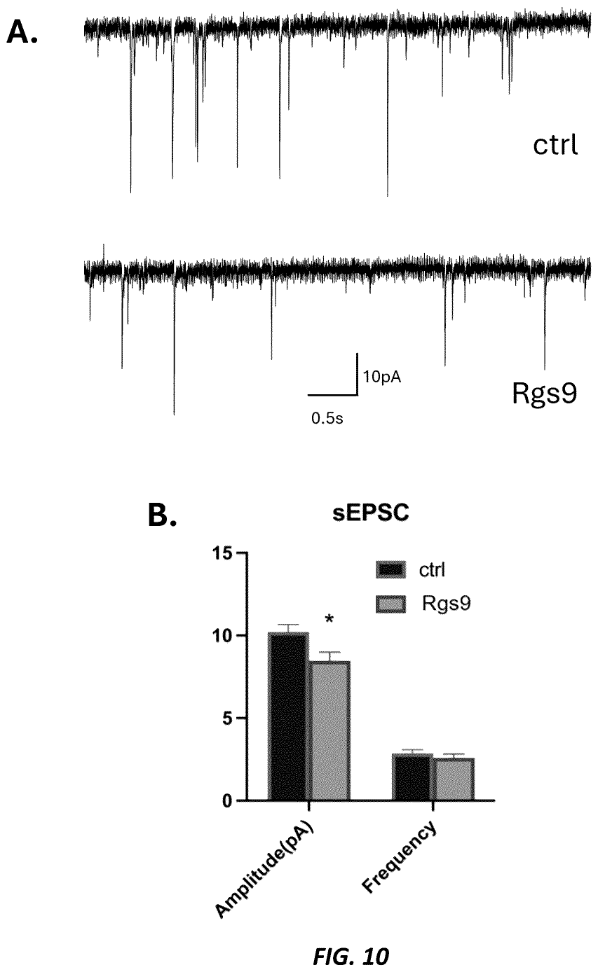

We next performed the whole-cell voltage-clamp recording of the corticostriatal pathway medium spiny neurons (MSNs) and compared their firing properties using 11 control and 7 Rgs9-Cre^{+/-}Tor1a^{i-ΔE/ΔE} mutant male mice at an average age of 184 days (range: 61-296 days). MSNs comprise ˜95% of the neurons in the striatum. We compared the paired-pulse facilitation (PPF) between the control and Rgs9-Cre^{+/-}Tor1a^{i-ΔE/ΔE} mice by recording MSNs and stimulating the cortical tract before it enters the striatum. We found no changes in PPF in the mutant mice (20 ms interval: control, 31 cells, 0.95±0.06; mutant, 20 cells, 0.88±0.04; Z=1.00, p=0.32; 50 ms interval: control, 30 cells, 0.91±0.07; mutant, 17 cells, 0.88±0.04; Z=0.39, p=0.69), which suggests normal short-term plasticity at the corticostriatal terminals to MSNs. However, there was a significant decrease in the amplitude of the spontaneous excitatory postsynaptic current (sEPSC) of MSNs (control, 27 cells, 10.22±0.44; mutant, 17 cells, 8.47±0.53; Z=2.54,p=0.011), while the frequency was not changed (control, 2.87±0.23 Hz; mutant, 2.60±0.23; Z=0.83, p=0.41, FIG. 10; Panels A and B).

Next, the intrinsic membrane properties were measured in whole-cell recording mode. The resting membrane properties of the MSNs were characterized in the brain slices from control (27 cells) and mutant mice (16 cells). As shown in Table 2, there was no significant difference in the resting membrane potential (RMP, mV) and the membrane resistance (MR) between control and mutant mice. However, the capacitance was significantly decreased, suggesting that mutant cells are of a smaller size.

TABLE 2
Intrinsic Membrane Properties of MSNs

| Genotype | Capacitance | MR | tau | RMP |
|---|---|---|---|---|
| Control | 60.7 ± 3.6 | 105 ± 13 | 1.07 ± 0.09 | −93.4 ± 0.4 |
| Rgs9 KI | 50.8 ± 3.5 | 121 ± 8 | 0.85 ± 0.08 | −92.5 ± 0.7 |
| Z score | 1.97 | −1.03 | 1.87 | 1.27 |
| p value | 0.048 | 0.30 | 0.06 | 0.21 |

Finally, the intrinsic excitability of the MSNs in the brain slices was measured with current step injections. The recorded neurons showed typical electrophysiological responses of the MSNs. The number of action potentials fired overall (control, 12.2±0.8; mutant, 11.5±0.8; Z=0.64, p=0.53) and at each current step (data not shown) were similar between control and mutant mice, suggesting intrinsic excitability is not altered.

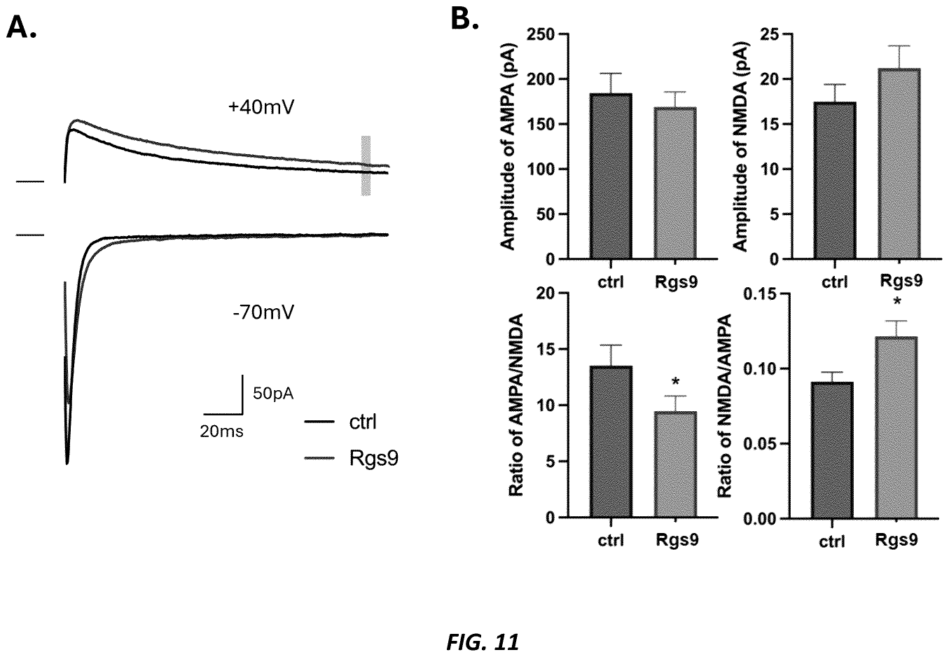

To determine whether the synaptic transmission is altered in the striatum, we next performed the whole-cell voltage-clamp recording of the MSNs and compared evoked excitatory postsynaptic currents (eEPSCs). Recordings in the dorsolateral striatum were stimulated using a tungsten bipolar electrode, placed dorsal to the recorded cell, and at the border of the corpus callosum and striatum. Electrical pulse durations were 50-100 ms. N-Methyl-D-aspartate/α-amino-3-hydroxy-5-methyl-4-isoxazole propionic acid (NMDA/AMPA) ratio was measured by evoking EPSCs while holding the cell at −70 mV and later at +40 mV. Peak amplitudes at −70 mV were used as the measure for the AMPA component. The NMDA component was measured 150 ms after the response onset at +40 mV (see, shaded box on the representative trace in FIG. 11; Panel A). We used the same 17 control and 7 mutant male mice as in the experiments described above. Although there were no differences in the AMPA currents (control, 27 cells, 184.4±22.0 pA; mutant, 13 cells, 169.1±16.6; Z=0.55, p=0.58) and NMDA currents (control, 17.5±1.9 pA; mutant, 21.2.0±2.5; Z=−1.16, p=0.25) themselves, as shown in FIG. 11, Panel B, the NMDA/AMPA ratio was significantly increased (control, 0.091±0.006; mutant, 0.122±0.010; Z=−2.52, p=0.012), and the AMPA/NMDA ratio was significantly decreased (control, 13.5±1.8; mutant, 9.5±1.3; Z=2.90, p=0.0038). Decreased normalized AMPA currents are consistent with the decreased sEPSC amplitude described above. Taken together, our electrophysiological analysis of corticostriatal pathway revealed the likely cellular basis for the overt dystonia present in this line of mice.

We claim:

1. A genetically modified mouse whose genome comprises a genetically modified Tor1a locus, wherein the genetically modified Tor1a locus comprises:
(i) a Tor1a allele comprising a ΔGAG deletion associated with DYT1 dystonia in exon 5; and
(ii) an inducible Tor1a allele comprising (a) loxP sites flanking a wild type exon 5 of Tor1a and (b) a downstream copy of exon 5 comprising the ΔGAG deletion associated with DYT1 dystonia.

2. A progeny mouse obtained by breeding the mouse comprising the genetically modified Tor1a locus of claim 1 with a transgenic mouse whose genome comprises a Cre recombinase,
wherein, in cells of the progeny mouse that express the Cre recombinase, the wild type copy of exon 5 of Tor1a in the inducible allele is deleted and the downstream copy of exon 5 comprising the ΔGAG deletion is expressed.

3. The progeny mouse of claim 2, wherein the Cre recombinase is expressed under the control of a tissue-specific promoter selected from the group consisting of Emx1 and Rgs9.

4. The progeny mouse of claim 3, wherein the tissue-specific promoter is Emx1, and the mouse exhibits overt dystonia.

5. The progeny mouse of claim 4, wherein the mouse does not exhibit impaired growth, reduced weight, seizures, neurodegeneration, or reduced lifespan.

6. The progeny mouse of claim 3, wherein the tissue-specific promoter is Rgs9, and the mouse exhibits overt dystonia.

7. The progeny mouse of claim 6, wherein the mouse does not exhibit impaired growth, reduced weight, seizures, neurodegeneration, or reduced lifespan.

8. The genetically modified mouse of claim 1, further comprising a polynucleotide encoding a Cre recombinase in one or more cells, wherein the mouse exhibits overt dystonia.

9. The genetically modified mouse of claim 8, wherein the mouse does not exhibit impaired growth, reduced weight, seizures, neurodegeneration, or reduced lifespan.

10. The genetically modified mouse of claim 8, wherein the polynucleotide is introduced into the genetically modified mouse via a gene therapy vector.

11. The genetically modified mouse of claim 10, wherein the gene therapy vector is selected from the group consisting of a naked polynucleotide, a polynucleotide complex, and a viral vector.

12. The genetically modified mouse of claim 11, wherein the gene therapy vector is a polynucleotide complex, and wherein the polynucleotide complex is a lipid nanoparticle comprising the polynucleotide and lipids.

13. The genetically modified mouse of claim 11, wherein the gene therapy vector is a viral vector, and wherein the viral vector is selected from the group consisting of a retrovirus, an adenovirus, a herpes simplex virus, a pox virus, a vaccinia virus, a lentivirus, and an adeno-associated virus (AAV).

14. The genetically modified mouse of claim 13, wherein the viral vector is pseudotyped to a specific tissue.

15. The genetically modified mouse of claim 14, wherein the specific tissue is the central and/or peripheral nervous system.

16. The genetically modified mouse of claim 8, wherein the polynucleotide is genomically integrated.

17. The genetically modified mouse of claim 8, wherein the polynucleotide is extrachromosomal.

18. A method of making a progeny mouse, comprising breeding:
I. a first mouse whose genome comprises a genetically modified Tor1a locus, wherein the genetically modified Tor1a locus comprises:
(i) a Tor1a allele comprising a ΔGAG deletion associated with DYT1 dystonia in exon 5; and
(ii) an inducible Tor1a allele comprising (a) loxP sites flanking a wild type exon 5 of Tor1a and (b) a downstream copy of exon 5 comprising the ΔGAG deletion associated with DYT1 dystonia; with
II. a second mouse whose genome comprises a Cre recombinase;
such that a progeny mouse whose genome comprises the ΔGAG deletion associated with DYT1 dystonia in exon 5 in both alleles of the Tor1a locus in one or more cells or tissues is obtained,
wherein the progeny mouse exhibits overt dystonia.

19. A method for screening a potential therapeutic agent to treat DYT1 dystonia, comprising:
I. obtaining the progeny mouse of claim 2;
II. administering the potential therapeutic agent to the progeny mouse; and
II. assaying one or more behavioral characteristics associated with DYT1 dystonia in the progeny mouse,
wherein a decrease in the one or more behavioral characteristics compared to a progeny mouse not receiving the potential therapeutic agent is indicative of an effective therapeutic agent.

20. A method for screening a potential therapeutic agent to treat DYT1 dystonia, comprising:
I. obtaining the genetically modified mouse of claim 10;
II. administering the potential therapeutic agent to the genetically modified mouse; and
II. assaying one or more behavioral characteristics associated with DYT1 dystonia in the genetically modified mouse,
wherein a decrease in the one or more behavioral characteristics compared to a genetically modified mouse not receiving the potential therapeutic agent is indicative of an effective therapeutic agent.
